(19) United States
(12) Patent Application Publication
(10) Pub. No.: US 2010/0312105 A1
(43) Pub. Date: Dec. 9, 2010

(54) METHODS FOR DIAGNOSING AND MONITORING TREATMENT OF LEWY BODY DEMENTIA BY ASSESSING DOPAMINE TRANSPORTER LEVEL

(75) Inventor: Mark Hurtt, Wallingford, CT (US)

Correspondence Address: RISSMAN HENDRICKS & OLIVERIO, LLP, 100 Cambridge Street, Suite 2101, BOSTON, MA, 02114, US

(73) Assignee: ALSERES PHARMACEUTICALS, INC., Hopkinton, MA (US)

(21) Appl. No.: 12/739,220

(22) PCT Filed: Oct. 29, 2008
(86) PCT No.: PCT/US08/81569

§ 371 Date: Aug. 26, 2010

Related U.S. Application Data
(60) Provisional application No. 60/984,194, filed on Oct. 31, 2007.

(51) Int. Cl. A61B 6/00

(52) U.S. Cl. 600/431

(57) ABSTRACT

A method of diagnosing Lewy Body Dementia in a human patient by assessing the level of dopamine transporter in at least one region of the patient's central nervous system, where a lowered level of dopamine transporter in the patient is indicative of Lewy Body Dementia. In embodiments of the invention, assessment of dopamine transporter levels includes assessing binding of a dopamine transporter ligand to the dopamine transporters using PET or SPECT.

### RELATED CASE INFORMATION

This application claims benefit of U.S. Provisional Application No. 60/984,194, filed Oct. 31, 2007. The teachings of the above application are incorporated herein by reference in its entirety.

### FIELD OF THE INVENTION

The present invention relates to the dopamine transporter, to imaging the dopamine transporter, and to diagnosing and monitoring Lewy Body Dementia.

### BACKGROUND OF THE INVENTION

Lewy Body Dementia, also referred to as Dementia with Lewy Bodies (DLB), is the second most frequent cause of degenerative dementia in elderly adults after Alzheimer's disease, and the second most frequent cause of hospitalization for dementia, after Alzheimer's disease. DLB is a neurodegenerative disorder associated with abnormal structures (Lewy bodies) found in certain areas of the brain. Lewy bodies (LB) are intracytoplasmic, spherical, eosinophilic neuronal inclusion bodies. The areas of predilection for LB are brainstem, subcortical nuclei, limbic cortex and neocortex. Their accumulation results in a loss of functional dopaminergic neuron terminals in the striatum.

Diagnosis of DLB using the current standards is inherently subjective, leading to inconsistent diagnosis. The diagnosis of DLB requires thorough clinical assessment including a detailed medical history and a full mental state, cognitive and physical examination by a clinician experienced in dementia. DLB patients have specific treatment requirements and functional disabilities that differ from those with other forms of dementia and that require specialized, often multi-disciplinary, treatment.

Fluctuating cognition, hallucinations and/or sleep disorders, which are infrequent in Alzheimer's disease (AD) and vascular dementia (VaD) patients, are present in DLB. These can be particularly disturbing to the DLB patient and their caregivers. DLB patients may deteriorate more quickly and/or require more intensive and more specialized care than do Alzheimer's patients.

Lewy bodies intracytoplasmic neuronal inclusions are intensely eosinophilic with routine haematoxylineosin staining They occur in the pigmented nuclei of the brainstem: in the substantia nigra and locus coeruleus, as well as in the motor nucleus of the vagus, in the nucleus basalis of Meynert, in the spinal cord and in sympathetic ganglia. Their structure and composition vary in different parts of the brain; in the brainstem they have a clearly defined halo which is usually absent from the cortical inclusions. Ultrastructurally they are chiefly composed of filaments with a greater central density region known as the core. Immunocytochemically, Lewy bodies share epitopes with phosphorylated and non-phosphorylated neurofilament subunits, tubulin, microtubule-associated proteins 1 and 2, and they positively immunostain with ubiqiutin.

Current estimates are that about 60-75% of diagnosed dementias are of the Alzheimer's and mixed (Alzheimer's and vascular dementia) type, 10-15% are Lewy Bodies type, with the remaining types being comprised of an entire spectrum of dementias including fronto-temporal lobar degeneration, alcoholic dementia, pure vascular dementia, etc.

Without being bound by any particular theory, DLB exhibits clinical overlap between Alzheimer's disease and Parkinson's disease. Noted are a loss of dopamine-producing neurons (in the substantia nigra) similar to that seen in Parkinson's disease, and a loss of acetylcholine-producing neurons (in the basal nucleus of Meynert and elsewhere) similar to that seen in Alzheimer's disease. Cerebral atrophy (or shrinkage) also occurs as the cerebral cortex degenerates. Autopsy series have revealed that the pathology of DLB is often concomitant with the pathology of Alzheimer's disease. That is, when Lewy Body inclusions are found in the cortex, they often co-occur with Alzheimer's disease pathology found primarily in the hippocampus, including: neurofibrillary tangles (abnormally phosphorylated tau protein), senile plaques (deposited beta-amyloid protein), and granulovacuolar degeneration.

Within DLB, the loss of cholinergic (acetylcholine-producing) neurons is thought to account for the degradation of cognitive and emotional functioning as in Alzheimer's disease, while the loss of dopaminergic (dopamine-producing) neurons is thought to account for the degradation of motor control as in Parkinson's disease. Thus, DLB is similar in some ways to both the dementia resulting from Alzheimer's disease and Parkinson's disease. In fact, it is often confused in its early stages with Alzheimer's disease and/or vascular dementia (multi-infarct dementia). The overlap of neuropathologies and presenting symptoms (cognitive, emotional, and motor) may make an accurate differential diagnosis difficult to reach.

In some instances, neuropathology shows Lewy body formation and selective neuronal loss in the brainstem and other subcortical nuclei, and Lewy bodies in the neocortex and limbic cortex, but at a frequency well below that reported for diffuse Lewy body disease. Senile plaques are common, whilst tangles are rare; the plaques tend to be the diffuse type without a neuritic component. These cases are similar to those reported as Lewy body variant of Alzheimer's disease, characterized by cortical and subcortical Lewy bodies, senile plaque formation and spongiform vacuolation of the temporal cortex.

It is to be understood that clinical indicia will vary among practitioners, over time, and with increasing knowledge. A common element to these or related diagnoses, will be a means to, in vivo, determine the number and location of Lewy bodies or the absence of Lewy bodies. In addition, the ability to determine, in vivo, the progression, regression, or status of Lewy bodies over time and/or in response to therapeutic treatment and environmental factors will be of great benefit. Furthermore, beyond making such determinations in human subjects, making such determinations in animal models will accelerate treatment research.

Gaig, et al., “¹²³I-Ioflupane SPECT in the diagnosis of suspected psychogenic Parkinsonism,” Movement Disorders, 21(11), 1994-1998 (2006); Costa, et al., “Dementia with Lewy bodies versus Alzheimer's disease: Role of dopamine transporter imaging,” Movement Disorders, 18(S7), S34-S38 (2004); and Walker et al., “Dementia with lewy bodies: A comparison of clinical diagnosis, FP-CIT SPECT imaging and autopsy” J Neurol Neurosurg Psychiatry 17353255 (Mar. 12, 2007) are incorporated herein by reference in their entirety as are all publications cited herein. Iopflupane or FP-CIT is also named ¹²³I-2β-carbomethoxy-3β-(4-iodophenyl)-N-(3-fluoropropyl)nortropane.

### SUMMARY OF THE INVENTION

We have discovered that abnormal levels of the dopamine transporter in human brain is indicative of Lewy Body Dementia. Assessing dopamine levels in the brain, therefore, can confirm a diagnosis of Lewy Body Dementia, or can assist in monitoring treatment of Lewy Body Dementia.

This invention employs ¹²³I compounds as disclosed in U.S. Pat. No. 5,493,026, with particular reference to 2β-carbomethoxy-3β-(4′-fluorophenyl)-N-(3-iodo-E-allyl) nortropane (ALTROPANE®, Alseres Pharmaceuticals, Inc. Hopkinton, Mass.) to help differentiate probable Dementia with Lewy Bodies from Alzheimer's disease and/or Parkinson's disease dementia. For these same functions, this invention also employs tropanes incorporating technetium (^{99m}Tc) as a radiolabel as disclosed in U.S. Pat. No. 6,171,576 and U.S. Pat. No. 6,548,041.

Various dopamine transporter imaging agents can be used to assay the dopamine transporter as a biological marker for Lewy Body Dementia. Such imaging is used to diagnose Lewy Body Dementia and to monitor it, e.g., as the patient matures and/or is treated over time.

The present invention provides methods of diagnosing Lewy Body Dementia in a human patient by assessing or determining dopamine transporter activity in at least one region of said patient's central nervous system.

In one aspect, the method comprises administering to the patient a labeled (i.e., radiolabeled) dopamine transporter ligand and the assessment comprises determining the amount of labeled dopamine transporter ligand that is bound to dopamine transporter. The amount of labeled dopamine transporter ligand that is bound to dopamine transporter is compared with a control. An elevated level of dopamine transporter in said patient is indicative of Lewy Body Dementia. PET or SPECT imaging are optional assessment techniques. The dopamine transporter ligand comprises a compound that binds to the dopamine transporter. Examples of suitable ligands include (¹¹C)CFT ((¹¹C)WIN 35,428), ¹²³I-Altropane®, and (¹⁸F)CFT. Ligands particularly suitable for use in PET include but are not limited to, (¹¹C) Altropane. Ligands suitable for use in SPECT include, but are not limited to, technetium-labeled phenyltropane probes, such as (^{99m}Tc) technepine, O-1505, and similar compounds. Other examples of compounds useful in the methods of the present invention are described in U.S. Pat. Nos. 5,493,026; 5,506,359; 5,770,180; 5,853,696; 5,948,933; 6,171,576; 6,548,041 and 7,081,238, the disclosures of which are hereby incorporated by reference. Reference is also made to U.S. Pat. No. 6,180,083 to Maculaire et al. disclosing

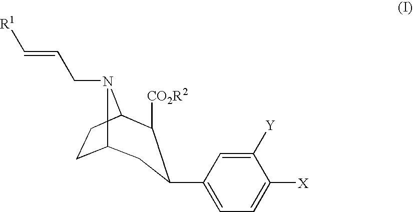

wherein R¹ represents I, a radioactive isotope of I or a group with the formula Sn(R³)₃ in which R³ is an alkyl group; R² represents H, a C₁ to C₆ alkyl group, a phenyl group, a phenyl group substituted by a halogen atom, a methyl group or a methoxy group, a phenylalkyl or phenylalkenyl group whose alkyl or alkenyl group comprises 1 to 6 carbon atoms and whose phenyl group may be substituted by a halogen atom, a C₃ to C₈ cycloalkyl group or an alkynyl group; wherein X represents Cl or F; and Y represents CH₃.

The portion of the patient's central nervous system for assessment is preferably a portion of the human brain, e.g., the striatum.

Assessing dopamine transporter to determine dopamine transporter levels can include assessing dopamine transporter availability or binding potential. For example, in a method wherein dopamine transporter availability is assessed, dopamine transporter availability in a patient is compared with the dopamine transporter availability in a control, wherein a lower dopamine transporter availability in the patient is indicative of Lewy Body Dementia. Similarly, when dopamine transporter binding potential is measured the dopamine transporter binding potential in the patient is compared with the dopamine transporter binding potential in a control, and a lower dopamine transporter binding potential in the patient is indicative of Lewy Body Dementia.

The present invention also provides a method of determining the effectiveness of a Lewy Body Dementia treatment for a human patient. The method includes determining or assessing an initial dopamine transporter level in at least one region of the patient's central nervous system, treating the patient and then determining or assessing dopamine transporter levels in the same region, e.g., after two or more weeks of treatment. The initial and subsequent dopamine transporter levels are then compared to determine or assess the effectiveness of treatment. A reduction in the rate of decline in dopamine transporter levels indicates that a treatment is effective. In one embodiment, a labeled dopamine transporter ligand is administered to the patient before assessing the initial dopamine transporter level and, if necessary, also before assessing the subsequent dopamine transporter level. In this method, the assessment comprises determining the amount of labeled dopamine transporter ligand that is bound to dopamine transporter. The subsequent step of assessing dopamine transporter levels can be repeated more than one time, in order to follow the course of treatment.

The treatment of Lewy Body Dementia can include, for example, administration of a pharmaceutical, such as rivastigmine (Novartis AG), donepezil (Eisai Co Ltd), and tacrine (Warner-Lambert Co) and Namenda® (Mematine HCl, Forest Pharmaceuticals). The assessment of effectiveness can include imaging by PET or SPECT techniques.

The effectiveness of a treatment can be determined by assessing dopamine transporter availability before treatment, and comparing this value with the dopamine transporter availability in subsequent assessment steps. A higher dopamine transporter availability, or a decreased rate of decline in dopamine transporter binding potential in the subsequent assessment, indicates that the treatment is effective. Similarly, the binding potential can be used to assess dopamine transporter levels, where the dopamine transporter binding potential in the initial assessment is compared with the binding potential in the subsequent assessment, and a higher dopamine transporter binding potential or a decreased rate of decline in dopamine transporter binding potential in subsequent assessments indicates that the treatment is effective.

The invention also provides a method of determining whether an individual has an early incidence of Lewy Body formation which may lead to Lewy Body dementia. The method includes assessing the level of dopamine transporter in at least one region of the patient's central nervous system and comparing the patient's dopamine transporter level to a predetermined normal dopamine transporter level. A lower than normal level is indicative of a increased probability of having Lewy Body Dementia. A labeled dopamine transporter ligand is administered before the assessing step, and the assessment step comprises determining the amount of labeled dopamine transporter ligand that is bound to dopamine transporter.

The invention further provides a method of monitoring the progress of a treatment for Lewy Body Dementia in a human patient. The method includes determining or assessing the level of dopamine transporter in at least one region of the patient's central nervous system a plurality of times during the treatment. Comparing the results of dopamine transporter level in the same region of the brain at various times during treatment enables one to monitor the progress of treatment. In this method, a labeled dopamine transporter ligand may be administered to the patient and the dopamine transporter level is assessed by measuring the amount of labeled dopamine transporter ligand that is bound to dopamine transporter. The amount of bound labeled dopamine transporter ligand is measured by any method of imaging, preferably using PET or SPECT imaging.

The methods of the present invention can provide one or more of the following advantages. For example, assessing dopamine transporter levels allows an objective, biologically based diagnosis of Lewy Body Dementia. Diagnosis based on dopamine transporter levels can be used for patients of all ages and both sexes. The method of the present invention are useful in diagnosing Lewy Body Dementia in adults, as well as in children. In one embodiment, imaging agents used to assess dopamine transporter levels including, but not limited to, ¹²³I-Altropane, are safe and well tolerated by patients.

Other features and advantages of the invention will be apparent to those skilled in the arts from the following description of the preferred embodiments and from the claims.

As used herein, the term “dopamine transporter ligand” means a compound that binds to the dopamine transporter. In one embodiment, compounds bind selectively to the dopamine transporter in preference to the serotonin transporter.

### DETAILED DESCRIPTION OF THE INVENTION

Assessing dopamine transporter levels are performed by assessing dopamine transporter availability using, e.g., PET (positron emission tomography) or SPECT (single photon emission computed tomography). To measure dopamine transporter availability, a labeled probe that targets the transporter is introduced into the brain, e.g., intravenously, and PET or SPECT is performed. From the PET or SPECT images, the density of the dopamine transporter is quantified by measuring the binding potential, where binding potential is defined as the maximum number of binding sites, Bₘₐₓ, divided by a dissociation constant, K_{d}. The binding potential is calculated from a continuous scan starting at about 15 minutes after introduction of the probe. The region of interest is identified and the counts in that region are determined. Using appropriate modeling, numerical values for binding potential are calculated and these values are compared between subjects who have undergone equivalent treatment and scanning protocols. The striatal binding potential of ¹²³I-Altropane (k3/k4) is calculated by the reference region approach as described by Farde, et al. (Farde, et al., 1989, J. Cereb. Blood Flow Metab 9:696 708).

For clarity, the dopamine transporter (also dopamine active transporter, DAT, SLC6A3) is a membrane spanning protein that binds the neurotransmitter dopamine and moves it from the synapse into a neuron. Imaging, as related to dopamine transporter, is a determination of the density of dopamine transporter in a region; e.g., a region of the brain.

Imaging agents that target the dopamine transporter include (¹¹C)Altropane, (¹¹C or ¹⁸F) WIN 35,428 ((¹¹C)CFT), ¹²³I-Altropane, (^{99m}Tc) O-1505, (^{99m}Tc) technepine, and similar compounds. These agents bind the dopamine transporter with varying affinities, allowing multiple, dissimilar assessments to be performed. Structures, synthesis, and/or sources of some of the above agents are described in, e.g., Fischman et al., 1998, Synapse 29:125 41 (¹²³I-Altropane); Madras et al., 1996, Synapse 22:239 46; Meltzer et al., 1993, J. Med. Chem. 36:855 62; and Milius et al., 1990, J. Medicinal Chem. 34:1728 31, each of which is incorporated herein by reference. Another useful compound includes ¹²³I Ioflupane (DatSCAN) from Nycomed-Amersham.

Compounds that are useful as imaging agents in the methods of the present invention include compounds described in U.S. Pat. No. 6,548,041, which is incorporated herein by reference in its entirety. These compounds have a tropane moiety linked through the nitrogen atom at the 8-position to a chelating ligand capable of complexing a technetium or rhenium radionuclide to produce a neutral labeled complex that selectively binds to a dopamine transporter. It is the tropane moiety which binds to the dopamine transporter.

Assessment of dopamine transporter levels can complement, and in some cases, supplant, traditional Lewy Body Dementia diagnostic techniques. The dopamine transporter level assessments using PET or SPECT provide objective, biological criteria for diagnosing Lewy Body Dementia, and can be used to confirm a Lewy Body Dementia diagnosis under the International Consensus Criteria (ICC) standard. (See McKeith I G, et al. “Consensus guidelines for the clinical and pathologic diagnosis of dementia with Lewy bodies,” Neurology 1996; 47:1113-1114, McKeith I G, et al. “Diagnosis and management of dementia with Lewy bodies,” Neurology 2005; 65(12):1863-72, and McKeith I G, et al. “Prospective validation of consensus criteria for the diagnosis of dementia with Lewy bodies,” Neurology. 2000a; 54:1050-8, each of which are incorporated herein by reference in their entirety. The assessments can also be used to resolve conflicting diagnoses, or to call into question a diagnosis or non-diagnosis of Lewy Body Dementia. Dopamine transport assessments are also used to refine subjective testing criteria for Lewy Body Dementia.

The ICC already include low dopamine transporter uptake in basal ganglia demonstrated by SPECT or PET imaging as a suggestive feature of DLB. If one or more of the suggestive features is present in the presence of one or more core features (fluctuating cognition, recurrent visual hallucinations and spontaneous features of Parkinsonism), a diagnosis of probable DLB is made. The ICC criteria for probable DLB, which have been prospectively validated on the basis of post-mortem data, have demonstrated a sensitivity of 83% and a specificity of 95%.

In addition, PET and SPECT imaging of the dopamine transporter can be used to monitor and adjust treatment of Lewy Body Dementia. The effectiveness of treatment for a particular patient is monitored by assessing dopamine transporter levels both before and after administration. For example, dopamine transporter levels is assessed immediately before a treatment, and then, e.g., two weeks, months, or longer after administration of treatment. The decreased availability of dopamine transporter will manifest as decreased binding potential in the PET or SPECT images. Such objective data assists a physician in determining the most effective drug and the most effective dosage for a particular patient.

Dopamine transporter level assessments are also used to monitor treatment over the long term, and to help a physician and patient determine whether treatment affects transporter levels and whether treatment can be stopped.

Finally, dopamine transporter level assessments identify individuals at risk for Lewy Body Dementia. Patients found to have lowered dopamine transporter levels are referred for conventional Lewy Body Dementia testing.

### EXAMPLES

The following examples serve to illustrate certain useful embodiments and aspects of the present invention and are not to be construed as limiting the scope thereof. Alternative materials and methods are also utilized to obtain similar results.

### Example 1

SPECT imaging of the dopamine transporter with ¹²³I-Altropane is conducted on six subjects previously diagnosed with Lewy Body Dementia, and on control individuals without a diagnosis of Lewy Body Dementia. ¹²³I-Altropane, E-2β-carbomethoxy-3β-(4-fluorophenyl)-N-1(1-iodoprop-1-en-3) nortropane, is an iodo analog of N-allyl CFT (WIN 35,428), a phenyltropane analog. The molecular formula of Altropane is C₁₈H₂₁IFNO₂. Before administration to human subjects or patients, Altropane is labeled with ¹²³I-, a gamma-emitting isotope with a half-life of 13.2 hours. For each individual tested, greater than 1 mCi of ¹²³I-Altropane is administered by intravenous injection at the onset of imaging. Images of the striatum are collected and analyzed by a radiologist to determine striatal binding potentials. In general, the methodology used for the SPECT imaging is the same as the methods described in Fischman et al., 1998, Synapse 29:125 41, which is incorporated herein by reference.

As determined by the imaging, these six Lewy Body Dementia individuals show reduced binding potential and, therefore, reduced dopamine transporter levels compared to expected levels for aged matched normal individuals.

### Example 2

Pre-clinical in vivo and in vitro studies performed in monkeys demonstrate that Altropane preferentially binds to dopamine rich areas of the striatum with a density that is within the range reported for the dopamine transporter. Madras, et al., 1998, Synapse 29:105 115. Altropane has demonstrated high selectivity for the dopamine transporter, compared to the seratonin transporter. Madras, et al., 1998, Synapse 29:93 104. Further, in vitro binding studies demonstrated that Altropane binds to a specific high-affinity site on the dopamine transporter. Elmaleh, et al., 1996, J. Nucl. Med. 37:1197 1202; Madras, et al., 1998, Synapse 29:116 127.

Diagnostic Assessments:

Each subject undergoes a standardized clinical assessment, as described by the ICC. This assessment includes a medical history and laboratory assessment to eliminate other causes of dementia. Any method for the diagnosis of Lewy Body Dementia may be used for comparison. The clinical evaluation is conducted by a clinician who knows and treats Lewy Body Dementia.

Subjects are given SSKI or Lugol's solution treatment to decrease thyroid exposure to ¹²³I.

Procedures:
1) Perform pre-injection Brief Neurological Assessment
2) Position the subject in the scanner with appropriate head immobilization.
3) Administer radiopharmaceutical over approximately thirty (30) seconds, followed by a 20 mL saline flush administered over approximately thirty (30) seconds, such that the total infusion time for the Altropane plus the saline flush is approximately sixty (60) seconds.
4) Acquire a series of consecutive two-minute SPECT scans.
5) Perform post-injection Brief Neurological Assessment approximately 60 to 90 minutes after ¹²³I-Altropane Administration and SPECT Imaging.

Approximately 8 mCi ¹²³I-Altropane is infused intravenously over approximately thirty (30) seconds, followed by a saline flush of 20 mL administered over approximately thirty (30) seconds, such that the total time of administration of the Altropane and saline flush is approximately sixty (60) seconds. Note that the volume for an 8 mCi injection can vary from approximately 5 to 20 mL.)

Each clinical dose of a sterile, pyrogen-free solution of ¹²³I-Altropane for intravenous (i.v.) injection contained:

| | | |
|---|---|---|
| ¹²³I--Altropane 8 mCi ¹²³I- | ~15 | ng Altropane. |
| Ethanol, U.S.P. | 7% | by volume |
| 0.9% Sodium Chloride for Injection, U.S.P. | 90% | by volume |
| Water for Injection, U.S.P. | 3% | by volume |

Effective head immobilization is useful for imaging. The orbital metal line is aligned with the plane of rotation. Dynamic SPECT imaging is begun immediately after completion of the infusion. Approximately fifteen (15) SPECT scans are acquired in sequence, starting immediately after the completion of ¹²³I-Altropane infusion. Each of the SPECT scans are acquired over a 2 minute period for a total of 60 minutes of imaging time, accounting for reset periods between each SPECT study.

As to scan times, it has been discovered that the signal-to-noise (S/N) ratio of the SPECT scans of the dopamine transporter ligand is optimal between the time of about 15 minutes and about 45 minutes post-injection of the dopamine transporter ligand.

Several alternatives are available for acquiring images. In one embodiment, the SPECT scanning is one continuous scan or a series of shorter scans. In another embodiment, the SPECT scanning comprises a series of one or more two-minute scans. In another embodiment, the SPECT scanning comprises a series of one or more ten-minute scans. In one particular embodiment, the SPECT images are generated using one continuous 30 minute scan. In another particular embodiment, the SPECT images are generated using a series of 2-minute scans for a period of 60 minutes. In another particular embodiment, the SPECT images are generated using a series of 4-minute scans for a period of 40 minutes.

A transverse slice set from each of the 15 SPECT scans is reconstructed using a Butterworth filter of order 4.0 and cut-off of 0.26 cycles/pixel as suggested starting points, or equivalent. Images are usefully optimized for each gamma camera used. Attenuation correction is performed using the Chang Algorithm.

Primary Analyses

A comparison is made between the Lewy Body Dementia and non-Lewy Body Dementia subjects with respect to baseline demographic and medical history data. For the quantitative variables the comparison use either a t-test or the Wilcoxon rank sum test, as appropriate. For the qualitative variables the comparison is based on Fisher's exact test.

Quantitative Analysis of ¹²³I-Altropane Images

The striatal binding potential of ¹²³I-Altropane (k3/k4) is calculated by the reference region approach as described by Farde, et al. (Farde, et al., 1989, J. Cereb. Blood Flow Metab 9:696 708). Briefly, specific binding to a receptor is a function of the density of receptors (Bₘₐₓ) and the dissociation constant of the ligand (K_{d}). Specific binding of the ligand reaches a maximum during the time span of the imaging procedure. The time of maximal specific binding is determined from time-activity curves (TAC) of specific and non-specific binding of the ligand. By assuming that non-specific binding is negligible in striatum and occipital cortex, the striatal time-activity curve (StrTAC) represents the kinetic behavior of specifically bound plus free ligand, while the occipital cortex TAC (OccTAC) represents the kinetic behavior of the free ligand only. Under these assumptions, the function, (StrTAC-OccTAC) defines the time dependence of bound ligand in the striatum. When this curve is fitted to a gamma variate function (A^{tn}e^{-mt}), and the maximum is divided by the value of the OccTAC at the same time, an equilibrium estimate of (k3/k4) is obtained.

Reconstructed SPECT scans are processed by a central reading facility. Transaxial images containing the striatum are summed at each time point using standardized criteria. Regions of interest (ROIs) are drawn around the left striatum, the right striatum, and a third ROI over the occipital cortex. TACs defined for average striatal and occipital cortex activity were used to calculate the striatal binding potential using the following formula: k3/k4=Max{StrTAC OccTAC}Time=t/{(OccTAC)}Time=t

The Binding Potential (BP) data collected from 24 patients is analysed. The results are tabulated.

¹²³I-Altropane is studied in several clinical trials in healthy volunteers, patients with Parkinson's Disease, patients with non-Parkinsonian movement disorders, adult patients with ADHD, and in Lewy Body Dementia. ¹²³I-Altropane, at doses of about 5 to about 8 mCi, (8 mCi is equivalent to 14.4 ng, or 34 pmol Altropane), ar used for the majority of studies.

The above study with single photon emission computed tomography (SPECT) using ¹²³I-Altropane demonstrates good correlation between increased striatal binding and the diagnosis of adult patients with Lewy Body Dementia. Thus, it appears that the methods of the present invention, e.g., using ¹²³I-Altropane SPECT, can provide an independent and objective diagnostic test that will complement the clinical diagnosis of Lewy Body Dementia.

### Example 3

### Participants

Twenty (20) adults having Lewy Body Dementia and 20 age-matched healthy control volunteers are used in the study.

Procedures

All participants are seen for three separate visits. During the first visit, written informed consent is obtained along with basic demographic data and medical/surgical history. Eligibility criteria are reviewed and established. Blood and urine samples are collected along with a 12-lead electrocardiogram. Some Lewy Body Dementia subjects take prescribed stimulants for management of their Lewy Body Dementia. With their physician's permission, these participants are removed from their medication for a four-week period prior to being scheduled for their SPECT scan.

During the second visit, scheduled for 15 weeks after the initial visit, the SPECT scan is conducted. Participants are evaluated at baseline for possible adverse events and all eligibility criteria are reviewed once again. All are then queried about their having taken the Lugol solution within the past 24 hours. Females then received a urine pregnancy test. Pre-injection vital signs and a brief neurological exam are conducted after which participants are positioned in the scanner. Over a 30 second period, the ¹²³I-Altropane is infused intravenously. A series of two-minute serial SPECT scans are then obtained for 60 minutes after which vital signs are again tested, the 12-lead ECG obtained again, and the brief neurological exam is repeated.

A third clinical visit is scheduled the following day at which time participants are interviewed about possible adverse events, a physical exam was conducted, vital signs and the 12-lead ECG are repeated, and a blood sample obtained.

SPECT Scan

While positioned horizontally in the scanner the ¹²³I-Altropane is injected. Serial two minute scans are acquired for a period of 60 minutes.

Time-activity curves (TAC) in the striatal regions (STR) are compared with areas in the occipital cortex (OCC) to calculate the time dependence of bound ¹²³I-Altropane STR minus (OCC). These data are fit to a gamma variate function and divided by the maximum OCC TAC to determine an equilibrium estimate of DAT binding potential (Bₘₐₓ/K_{d}). Measures of binding potential were then standardized to age 28.4 years for comparison between the Lewy Body Dementia and control groups.

Results

SPECT data are successfully obtained from 24 adults; 8 in the Lewy Body Dementia group and 16 in the control group. One-tailed t-test (unequal variance) revealed that the Lewy Body Dementia group have significantly lesser binding potential for the ¹²³I-Altropane than does the control group.

To determine the classification accuracy of the age-corrected binding potentials, a Binding Potential cutoff score is selected as being +1 SD above the normal mean for determination of Lewy Body Dementia diagnosis.

The present results show increased dopamine transporter density in striatum in adults with Lewy Body Dementia relative to an age-matched control group. Subjects with Lewy Body Dementia have lower ¹²³I-Altropane uptake in striatum than control adults. Moreover, altropane binding potentials are significantly related to degree of both inattention and hyperactive-impulsive symptoms, further solidifying the conclusion that lowered dopamine transporter density is associated with the degree of Lewy Body Dementia symptoms within this sample.

### Example 4

### Study design

This study is a multi-center, open-label, non-randomized, single dose clinical study to assess the diagnostic efficacy and safety of ¹²³I-Altropane in subjects with DLB.

The primary objective is to determine the diagnostic efficacy of the visual assessment of ¹²³I-Altropane®, SPECT images in differentiating between “probable DLB” and non-DLB subjects when compared to the clinical diagnosis established by a consensus panel (CP) as the standard. Secondary objectives include determining the positive and negative predictive values.

The absence of structural abnormalities in the basal ganglia are ruled out by cerebral magnetic resonance imaging (MRI) or computed tomography (CT) performed within 6 months prior to screening. The results need to be negative for vascular abnormalities indicative of infarction in the region of the basal ganglia.

The injection of ¹²³I-Altropane was open but clinical diagnosis and image analysis were blind.

Methodology

For the efficacy assessment, the results of the ¹²³I-Altropane image analysis are compared to the clinical diagnosis.

Study Population

The study population consists of demented subjects (between 55-90 years of age) with features of probable or possible DLB and subjects with features of non-DLB (e.g., AD or VaD). The DLB subjects are selected for screening from movement disorder clinic databases, dementia services, memory clinics, and other general neurology clinics. The distribution of evaluable DLB and non-DLB subjects are assessed on an ongoing basis during the study as determined by the clinical diagnosis of the on-site physician.

The subjects present positive assessment for dementia in accordance with the Diagnostic and Statistical Manual of Mental Disorder—Fourth Edition (DSM-IV) criteria and fulfilled at least one of the following: the ICC for probable or possible DLB, the NINCDS-ADRDA for AD, or the National Institute of Neurological Disorders and Stroke—Association Internationale Pour la Recherche et l'Enseignement en Neurosciences (NINDS-AIREN) for VaD. PDD patients were excluded (dementia occurring at least one year after PD diagnosis).

Clinical Diagnosis and “Standard of Truth”

The clinical diagnosis is established using the ICC and based on a standardized and comprehensive clinical and neuropsychiatric evaluation. The “standard of truth” or “gold standard” is the clinical diagnosis of DLB (“probable” or “possible”) versus non-DLB (probable or possible AD, probable or possible VaD) established by an independent CP (ICP) consisting of 3 internationally recognized experts in the diagnosis of dementia and in DLB in particular. Both “probable” and “possible” DLB patients are assumed to be DLB patients.

¹²³I-Altropane SPECT Imaging

¹²³I-Altropane SPECT images are obtained as recommended in the SPC. The images are acquired using a multi-headed (2- or 3-headed) gamma camera and imaging begins at about 15 minutes post injection and ends at about 45 minutes post injection.

Eight images are evaluated at an independent image review center (IRC) as part of a blind image evaluation (BIE) performed by 3 independent readers (nuclear physicians with expertise in neuroimaging). Images are evaluated both visually and by a semi-quantitative assessment (ROI). During visual assessment, each of 3 blind readers classify the images as normal, abnormal or other (an image that could not be assigned to one of the aforementioned classes) described below.

Normal images: Normal images are characterized by uptake of the tracer in both right and left putamen and caudate nuclei. The image is largely symmetrical with approximately equal levels of uptake on both left and right sides. Activity is contained close to the center of the image forming 2 crescent shaped areas of uptake.
Abnormal image type 1: Uptake is asymmetric with normal or almost normal putamen activity in 1 hemisphere and a more marked change on the other side.
Abnormal image type 2: Uptake is significantly reduced in the putamen on both the right and left sides. Activity is confined to the caudate nuclei and forms 2 roughly symmetrical, circular areas.
Abnormal image type 3: Uptake is virtually absent from both putamen and caudate nuclei on each side of the brain resulting in a significant reduction in contrast and the visualization of background activity throughout the rest of the image.
Other: Option is provided if an image cannot be assigned to any of the categories above.

The semi-quantitative assessment is a ROI-based analysis to determine the striatal DAT density calculated as the ratio of total specific striatal activity/non-specific activity. The striatal ROI data are analyzed by a reader to examine the whole striatal, caudate, and putamen uptake in each hemisphere. Analysis of the co-primary efficacy endpoints, sensitivity and specificity, is solely based on the division of the above classes into normal or abnormal based on the result of the BIE. The 3 independent blinded readers interpret the images individually, with the images being presented to the readers in random order. The readers are blinded to the subject's personal and clinical information except for the subject's age. Age is required for appropriate evaluation of the SPECT images because with increasing age, the nigrostriatal ¹²³I-Altropane uptake decreases and the non-specific uptake increases due to overall decreased circulatory capacity.

Efficacy Variables

The Co-Primary Efficacy Endpoints were Sensitivity and Specificity.

| | Evaluation of SPECT Images | | |
|---|---|---|---|
| Standard of truth | Abnormal | Normal | Total |
| Abnormal | True Positive (TP) | True Negative (TN) | TP + FN |
| (Probable DLB) | | | |
| Normal | False Positive (FP) | False Negative (FN) | FP + TN |
| (non-DLB) | | | |
| Total | TP + FP | FN + TN | N |

Sensitivity and specificity (with ¹²³I-Altropane SPECT visual assessments and Consensus Panel clinical diagnosis) were defined as follows:

Sensitivity=TP/(TP+FN) i.e. the percentage of times that the image diagnosis is DLB given that the clinical diagnosis is DLB.

Specificity=TN/(TN+FP) i.e. the percentage of times that the image diagnosis is non-DLB given that the clinical diagnosis is non-DLB.

Secondary Efficacy Endpoints
1. Accuracy=(TP+TN)/(TP+FP+TN+FN) i.e. the percentage of times the image diagnosis matched the clinical diagnosis,
   Positive Predictive Value (PPV)=TP/(TP+FP) i.e. the percentage of times that the clinical diagnosis is DLB given that the image diagnosis is DLB
   Negative Predictive Value (NPV)=TN/(TN+FN) i.e. the percentage of times that the clinical diagnosis is non-DLB given that the image diagnosis is non-DLB
2. Semi-quantitative analysis (ROI) of the altropane images to compare striatal uptake ratios of altropane between the 3 groups of probable, possible and non-DLB in specific regions of interest (i.e., striatum, caudate, and putamen in both hemispheres)
3. Assessment of the impact of altropane SPECT visual assessment findings of the on-site investigator's ability to establish a diagnosis, to make management decisions and evaluating the confidence of diagnosis by comparing pre- and post-imaging results
4. Summary of the proportions of abnormal altropane SPECT visual assessment findings in relation to the groups of probable DLB, possible DLB, and non-DLB as established by an independent CP

Statistical Analyses

For both diagnostic parameters, an exact 1-sided binomial test is used to test the null hypothesis H0: p≦p0. In this case, p0 represents a pre-defined threshold for sensitivity or specificity. The alternate hypothesis is given by H1: p>p0. The parameter p represents the sensitivity or specificity for an independent blinded reader's diagnosis with access to ¹²³I-Altropane SPECT imaging.

On-Site Clinical Diagnosis

The on-site clinical diagnosis is established by the investigator before and after the Altropane imaging. This diagnosis is based on all available cognitive, neuropsychiatric, neurological, and clinical data. After the baseline testing is completed, the investigator is asked to establish the diagnosis as to probable DLB, possible DLB, or other forms of dementia (e.g., AD, VaD) using internationally accepted diagnostic criteria (including the ICC). The on-site investigators are then asked for a final clinical diagnosis to be made on the basis of all available subject information—including Altropane image findings.

Comparison of Results in Subpopulations

Analyses for sensitivity, specificity, and accuracy are performed for the following subgroups: age, presence of Parkinsonism severe dementia/cognitive impairment, study center, and dose of radioactivity administered.

### Example 5

### ALTROPANE® SPECT Imaging in Lewy Body Dementia and Control Subjects

The following example summarizes single photon emission computed tomographic (SPECT) image acquisition, analysis methods for Lewy Body Dementia research participants and healthy controls participating in an ¹²³I-altropane SPECT imaging study. The experiment is an open label study evaluating time-activity curves and striatal dopamine transporter occupancy over the first hour post bolus injection of ¹²³I-altropane using temporally well-resolved dynamic SPECT in Lewy Body Dementia subjects and similarly aged healthy control subjects. In addition, for the Lewy Body Dementia subjects, a second injection and scan is performed on each subject on a separate test day to evaluate the test/retest reproducibility data for both visual and quantitative analyses.

### A. Subjects and Drug Dosing

Fifteen idiopathic Lewy Body Dementia subjects are recruited through local advertising and word-of-mouth for enrollment in the study. All fifteen subjects complete two separate injection and SPECT scan sessions with approximately 296 mBq (8 mCi) of ¹²³I-altropane. Subjects undergo assessment by a neurology specialist.

Twelve healthy controls subjects are recruited for participation in a single scan session with ¹²³I-altropane using the identical SPECT image acquisition protocol as the Lewy Body Dementia subjects. Healthy subjects are evaluated by a neurology specialist to confirm the absence of neurologic illness.

### B. SPECT Imaging and Analysis

SPECT scans are acquired for 60 min following the intravenous injection with 296 mBq (8.0 mCi) of ¹²³I altropane. A series of dynamic SPECT scans are obtained as five scans at 6 minutes per acquisition followed by three scans at 10 minutes per acquisition. The subject remains in the camera for the duration of the acquisition. Each SPECT study is acquired on a Philips PRISM 3000XP triple-headed SPECT camera (Cleveland, Ohio, USA) fitted with fan-beam collimators. Each head rotates 360 degrees, sampling every 3 degrees for a total of 120 raw projection images per head. Projection data are collected in a 128×128 matrix within a symmetric energy window centered at 159 kEv (+/−10%). This acquisition protocol permits the post hoc analysis of imaging data at each time point using information from 1, 2, or all 3 heads, hence modeling the impact of different injected doses of ¹²³I altropane at 2.7, 5.3, and 8.0 mCi, respectively.

Data are re-constructed using filtered back projection and a simple ramp filter followed by a post hoc (3-D) standardized low pass filter. Attenuation correction is performed applying a Chang 0 correction and a mu of 0.11 cm⁻¹ using a standard or custom software. Regions of interest (ROIs) are placed in the 3-head data scans with individual ROI sampling of the left and right caudate and putamen and an occipital background region. The ROIs are then applied to all images in the injection session (total of 24=8 time points×3 head conditions (1, 1&2, 1&2&3). To check for head movement within each scan session, five external skin fiducial markers containing 1 μCi of ¹²³I are placed along the canthomeatal line (2 right side, 3 left side) prior to each SPECT scan. Total counts within the ROI, total volume, and count density (counts/voxel) are extracted from each scan and logged in a data spreadsheet for determination of striatal uptake ratios (SBR) defined as the density of counts (counts per voxel per minute) in the striatal region divided by the density of counts in the occipital background region. The mean striatal SBR scores are calculated as the mean of the left and right caudate and putamen SBR scores. Since sampling in striatal sub-regions uses the identical size ROI, there is equal contribution from left and right caudate and putamen to the mean SBR.

The time-activity data for mean striatal count densities and occipital background for each of the 8 images acquired in the 60 minutes post injection sampling period are plotted to permit visual interrogation of the uptake and washout characteristics of ¹²³I-altropane in Lew Body Dementia and controls. In addition, each SBR ratio obtained at the 8 time points after injection are plotted. The peak SBR is assessed for each subject for data acquired using 1, 2, and all 3 heads of imaging data.

For each Lewy Body Dementia subject a composite SBR ratio is determined corresponding to data collected over about 13-40 minutes post injection (27 minutes total time). These time points are selected based on review of the peak and persistence of SBRs over the 60 minutes of image acquisition and a visual interrogation of the scan for optimal identification of striatal structures. This composite SBR is then compared within each subject's first and second ¹²³I-altropane injection days to review the reproducibility of the SBR using the following equation:

(composite SBR first injection−composite SBR second injection)/(composite SBR first injection).

### C. Results

All Lewy Body Dementia subjects enrolled complete the trial with a baseline and retest ¹²³I-altropane SPECT studies and all healthy controls complete all imaging assessments for a single study with ¹²³I altropane. All imaging data are included in the analysis.

### Dynamic Time Activity Data for ¹²³I Altropane in Lewy Body Dementia

Following bolus injection of ¹²³I-altropane in controls and Lewy Body Dementia subjects peak striatal count densities are noted at 10 minutes post injection. Without being bound by an particular belief, this is assumed in that most subjects rapidly eliminate altropane from the brain. Localization of uptake in striatal structures is well demonstrated in all subjects in the study with the characteristic “comma” shaped appearance of striatal uptake in controls and more asymmetric uptake (left-right asymmetry and caudate>putamen asymmetry) in Lewy Body Dementia subjects consistent with other dopamine transporter studies using altropane and other SPECT studies in Lewy Body Dementia. By 20-30 minutes post injection of ¹²³I-altropane rates of washout from striatum and occipital background region are similar resulting in stability of the striatal binding ratio.

Binding ratios decrease slightly when moving from 3 imaging heads of projection data to 2 imaging heads of projection data and further when using just one imaging head in the SPECT reconstruction. There is minimal effect in reducing the imaging heads from three to two on the shape of the SBR curves, with peak SBRs occurring from 15 minutes post injection for both the one and two head scenarios, suggesting an optimal scanning protocol from the perspective of signal to noise at the points with the highest binding ratios. Data indicates the peak SBRs for Lewy Body Dementia and control subjects (based on a single scan) demonstrating the small reductions in peak SBR when fewer imaging heads are incorporated into the reconstruction.

Examination of the semi-quantitative peak SBR demonstrates differences between the Lewy Body Dementia and healthy controls.

### Test-Retest Reproducibility of ¹²³I-Altropane Injection in Lewy Body Dementia

The test-retest reproducibility of the SPECT imaging is assessed in all 15 Lewy Body Dementia subjects using a mean total binding ratio taken as the mean of scan numbers 3 through six corresponding to roughly 30 minutes of imaging commencing about 15 minutes after bolus injection of ¹²³I-altropane. For this measure the percent test/retest reproducibility is defined as: (ratio test−ratio retest)/(ratio test). The results for all Lewy Body Dementia subjects under conditions of 3, 2, and 1 imaging head data reconstructions are summarized.

Total striatal binding is obtained during the time points corresponding to about 30 minutes commencing 15 minutes after ¹²³I-altropane injection demonstrates reproducibility in the 15 Lewy Body Dementia subjects studied.

### Incorporation by Reference

The patent and scientific literature referred to herein establishes knowledge that is available to those of skill in the art. All issued patents, patent applications, published foreign applications, and published references, which are cited herein, are hereby incorporated by reference to the same extent as if each was specifically and individually indicated to be incorporated by reference in their entirety.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of diagnosing Lewy Body Dementia in a human patient by the steps of:
(a) administering to a patient a labeled dopamine transporter ligand; and
(b) assessing the amount of said labeled dopamine transporter ligand that is bound to dopamine transporter in at least one region of said patient's central nervous system,
wherein a lowered level of said dopamine transporter in said patient is diagnostic of Lewy Body Dementia.

2. The method of claim 1, wherein said assessment is by SPECT imaging.

3. The method of claim 1, wherein the method further comprises the step of
(c) comparing the amount of labeled dopamine transporter ligand that is bound to dopamine transporter with a control.

4. The method of claim 1, wherein the dopamine transporter ligand comprises a compound that binds to the dopamine transporter.

5. The method of claim 1, wherein said ligand comprises ¹²³I-altropane and said assessment comprises imaging by SPECT.

6. The method of claim 1, wherein said at least one region of said patient's central nervous system comprises a portion of the brain.

7. The method of claim 6, wherein said portion of the brain comprises the striatum.

8. The method of claim 1, further comprising (c) comparing dopamine transporter availability with the dopamine transporter availability in a control, wherein a lower dopamine transporter availability in said patient is diagnostic of Lewy Body Dementia.

9. The method of claim 1, wherein a lower dopamine transporter binding potential in said patient is diagnostic of Lewy Body Dementia.

10. A method of determining whether an individual has an indicator of Lewy Body formation, the method comprising the steps of:
(a) administering to a patient a labeled dopamine transporter ligand; and
(b) assessing the amount of said labeled dopamine transporter ligand that is bound to dopamine transporter in at least one region of said patient's central nervous system,
wherein a lowered level of said dopamine transporter in said patient is diagnostic of a heightened probability of Lewy Body Dementia.

11. The method of claim 10, wherein the dopamine transporter ligand is ¹²³I-altropane.

12. A method of determining effectiveness of a therapeutic regimen on progression of Lewy Body formation or Lewy Body Dementia, the method comprising the steps of:
(a) at a first time point administering to a patient a labeled dopamine transporter ligand;
(b) assessing the amount of labeled dopamine transporter ligand that is bound to dopamine transporter in at least one region of said patient's central nervous system;
(c) at a second time point after said first time point administering to said patient a labeled dopamine transporter ligand; and
(d) assessing the amount of said labeled dopamine transporter ligand that is bound to dopamine transporter in the same at least one region of said patient's central nervous system; and,
(e) comparing the relative amounts of labeled dopamine transporter ligand that are bound to dopamine transporter in said at least one region of said patient's central nervous system,
wherein an increase in level of dopamine transporter in said patient in said at least one region, or a reduction in the rate of decline of dopamine transporter in said patient, is indicative of efficacy of said regimen.
